(19) United States
(12) Patent Application Publication
(10) Pub. No.: US 2009/0156616 A1
(43) Pub. Date: Jun. 18, 2009

(54) THERAPEUTIC AGENTS

(76) Inventors: Jonas Bostrom, Molndal (SE); Leifeng Cheng, Molndal (SE); Roine Olsson, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 11/996,117

(22) PCT Filed: Jul. 17, 2006
(86) PCT No.: PCT/GB06/02638

§ 371 Date: Aug. 11, 2008

(30) Foreign Application Priority Data
Jul. 19, 2005 (GB) 0514739.2

(51) Int. Cl. A61K 31/4965; C07D 231/14; A61K 31/415; C07D 241/24; A61K 31/455; C07D 213/82

(52) U.S. Cl. 514/255.06; 548/374.1; 514/406; 544/406; 514/355; 546/316

(57) ABSTRACT

The present invention relates to compounds of formula (I) and processes for preparing such compounds, their use in the treatment of obesity, psychiatric and neurological disorders, to methods for their therapeutic use and to pharmaceutical compositions containing them.

### FIELD OF INVENTION

The present invention relates to certain thioamide compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354). However, there is a need for CB₁ modulators with improved physicochemical properties and/or DMPK properties and/or pharmacodynamic properties.

### DESCRIPTION OF THE INVENTION

The invention relates to a compound of formula (I)

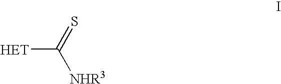

including pharmaceutically acceptable salts thereof wherein HET represents
a) a group of formula A

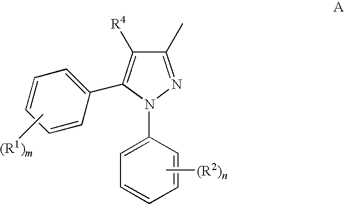

b) a group of formula B

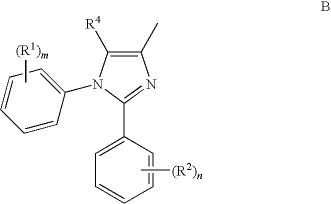

c) a group of formula C

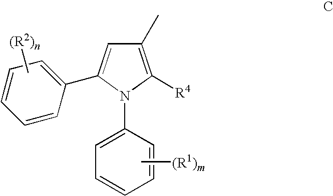

d) a group of formula D

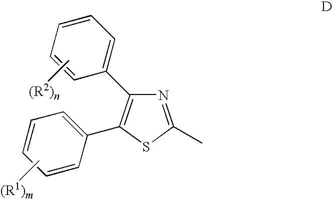

e) a group of formula E

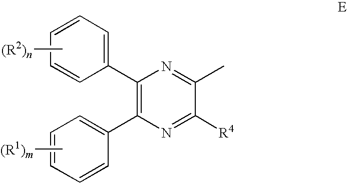

f) a group of formula F

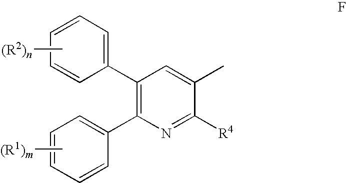

or g) a group of formula G

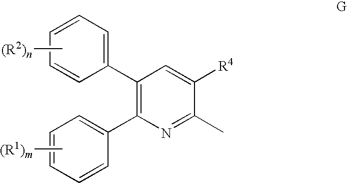

wherein
R¹ represents a) a C₁₋₁₀alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O in which R⁵ represents a C₁₋₁₀alkyl group optionally substituted by one or more fluoro or d) halo or e) a C₁₋₄alkyl group;
m is 0, 1, 2 or 3;
R² represents a C₁₋₃alkyl group, a C₁₋₃alkoxy group, hydroxy, nitro, cyano or halo
n is 0, 1, 2 or 3;
R³ represents a) cyclohexyl optionally substituted by one or more of the following: hydroxy, fluoro, amino, mono or di C₁₋₃alkylamino b) piperidino optionally substituted by one or more hydroxy c) phenyl optionally substituted by one or more of the following: hydroxy, halo or a C₁₋₄alkyl group d) pyridyl optionally substituted by a C₁₋₄alkyl group or
e) a C₄₋₉alkyl group; and
R⁴ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more hydroxy, fluoro, amino, monoC₁₋₄alkylamino, diC₁₋₄alkylamino or cyano.

In a first group of compounds of formula I, HET represents a group of formula A and R¹, R², R³, R⁴, n and m are as previously defined.

In a second group of compounds of formula I, HET represents a group of formula B and R¹, R², R³, R⁴, n and m are as previously defined.

In a third group of compounds of formula I, HET represents a group of formula C and R¹, R², R³, R⁴, n and m are as previously defined.

In a fourth group of compounds of formula I, HET represents a group of formula D and R¹, R², R³, R⁴, n and m are as previously defined.

In a fifth group of compounds of formula I, HET represents a group of formula E and R¹, R², R³, R⁴, n and m are as previously defined.

In a sixth group of compounds of formula I, HET represents a group of formula F and R¹, R², R³, R⁴, n and m are as previously defined.

In a seventh group of compounds of formula I, HET represents a group of formula G and R¹, R², R³, R⁴, n and m are as previously defined.

It will be understood that when m is 2 or 3 then the groups R^{a} are independently selected so that they may be the same or different and similarly when n is 2 or 3 then the groups R¹ are independently selected so that they may be the same or different.

Suitable groups in which R¹ represents a group R⁵S(O)₂O in which R⁵ represents a C₁₋₆alkyl group optionally substituted by one or more fluoro include methanesulfonyloxy, ethanesulfonyloxy, n-propylsulfonyloxy, n-butylsulfonyloxy, 3-methylbutane-1-sulfonyloxy, 3,3-dimethylbutane-1-sulfonyloxy, fluoromethylsulfonyloxy, difluoromethylsulfonyloxy, trifluoromethylsulfonyloxy, mono, di or tri (fluoroethyl)sulfonyloxy, 3,3,3-trifluoropropyl-1-sulfonyloxy, or 4,4,4-trifluorobutyl-1-sulfonyloxy,

Suitable groups in which R¹ represents a C₁₋₆alkoxy group optionally substituted by one or more fluoro include butoxy, pentyloxy, hexyloxy, fluoromethoxy, 3- fluoropropoxy, difluoromethoxy, trifluoroethoxy, 3,3,3-trifluoropropoxy, 4,4,4-trifluorobutoxy, 5,5,5,-trifluoropentyloxy and 6,6,6-trifluorohexyloxy.

Suitably R² represents a methyl, ethyl, propyl, methoxy, ethoxy, propoxy, hydroxy, nitro, cyano, chloro or fluoro.

Suitably R³ represents piperidino optionally substituted by one or more hydroxy or R³ represents cyclohexyl substituted by one or more of the following: hydroxy, fluoro, amino, mono or di C₁₋₃alkylamino, carboxy or a C₁₋₄alkoxycarbonyl group; for example 2-hydroxycyclohexyl, 3-hydroxycyclohexyl, 4-hydroxycyclohexyl, 2-aminocyclohexyl, 3-aminocyclohexyl, 2-dimethylaminocyclohexyl, 3-dimethylaminocyclohexyl or 4,4-difluorocyclohexyl. In one group of compounds the substituent is in the 2 or 3 position. In another group of compounds the substituent on the cyclohexyl ring is in the cis conformation with respect to the nitrogen of the amide. In another group of compounds the substituent on the cyclohexyl ring is in the trans conformation with respect to the nitrogen of the amide.

Suitably R⁴ represents H, cyano or methyl.

Specific compounds of the invention include one or more of the following:
3,3,3-Trifluoro-propane-1-sulfonic acid 4-[2-(2,4-dichloro-phenyl)-5-(4,4-difluorocyclohexylthiocarbamoyl)-4-methyl-2H-pyrazol-3-yl]-phenylester
3,3,3-Trifluoro-propane-1-sulfonic acid 4-[2-(2,4-dichloro-phenyl)-5-(2-hydroxycyclohexylthiocarbamoyl)-4-methyl-2H-pyrazol-3-yl]-phenyl ester
3,3,3-Trifluoro-propane-1-sulfonic acid 4-[2-(2,4-dichloro-phenyl)-5-(2-dimethylaminocyclohexylthiocarbamoyl)-4-methyl-2H-pyrazol-3-yl]-phenyl ester
3-Fluoro-propane-1-sulfonic acid 4-[2-(2,4-dichloro-phenyl)-5-(4,4-difluorocyclohexylthiocarbamoyl)-4-methyl-2H-pyrazol-3-yl]-phenyl ester
1-(2,4-Dichloro-phenyl)-4-methyl-5-[4-(3,3,3-trifluoro-propoxy)-phenyl]-1H-pyrazole-3-carbothioic acid (4,4-difluoro-cyclohexyl)-amide
1-(2,4-Dichloro-phenyl)-5-[4-(3-fluoro-propoxy)-phenyl]-4-methyl-1H-pyrazole-3-carbothioic acid (4,4-difluoro-cyclohexyl)-amide
3-Fluoro-propane-1-sulfonic acid 4-[2-(2,4-dichloro-phenyl)-5-(3-hydroxy-cyclohexylthiocarbamoyl)-4-methyl-2H-pyrazol-3-yl]-phenyl ester
3-Fluoro-propane-1-sulfonic acid 4-[5-(3-amino-cyclohexylthiocarbamoyl)-2-(2,4-dichloro-phenyl)-4-methyl-2H-pyrazol-3-yl]-phenyl ester
3-Fluoro-propane-1-sulfonic acid 4-[2-(2,4-dichloro-phenyl)-4-methyl-5-(5-trifluoromethyl-pyridin-2-ylthiocarbamoyl)-2H-pyrazol-3-yl]-phenyl ester
3,3,3-Trifluoro-propane-1-sulfonic acid 4-[2-(2,4-dichloro-phenyl)-4-(4,4-difluorocyclohexylthiocarbamoyl)-5-methyl-imidazol-1-yl]-phenyl ester
3,3,3-Trifluoro-propane-1-sulfonic acid 4-[2-(2,4-dichloro-phenyl)-4-(2-hydroxy-cyclohexylthiocarbamoyl)-5-methyl-imidazol-1-yl]-phenyl ester
3,3,3-Trifluoro-propane-1-sulfonic acid 4-[2-(2,4-dichloro-phenyl)-4-(2-dimethylamino-cyclohexylthiocarbamoyl)-5-methyl-imidazol-1-yl]-phenyl ester
3-Fluoro-propane-1-sulfonic acid 4-[2-(2,4-dichloro-phenyl)-4-(4,4-difluoro-cyclohexylthiocarbamoyl)-5-methyl-imidazol-1-yl]-phenyl ester
2-(2,4-Dichloro-phenyl)-5-methyl-1-[4-(3,3,3-trifluoro-propoxy)-phenyl]-1H-imidazole-4-carbothioic acid (4,4-difluoro-cyclohexyl)-amide
2-(2,4-Dichloro-phenyl)-1-[4-(3-fluoro-propoxy)-phenyl]-5-methyl-1H-imidazole-4-carbothioic acid (4,4-difluoro-cyclohexyl)-amide
3-Fluoro-propane-1-sulfonic acid 4-[2-(2,4-dichloro-phenyl)-4-(3-hydroxy-cyclohexylthiocarbamoyl)-5-methyl-imidazol-1-yl]-phenyl ester
3-Fluoro-propane-1-sulfonic acid 4-[4-(3-amino-cyclohexylthiocarbamoyl)-2-(2,4-dichloro-phenyl)-5-methyl-imidazol-1-yl]-phenyl ester
3-Fluoro-propane-1-sulfonic acid 4-[2-(2,4-dichloro-phenyl)-5-methyl-4-(5-trifluoromethyl-pyridin-2-ylthiocarbamoyl)-imidazol-1-yl]-phenyl ester
3-Fluoro-propane-1-sulfonic acid 4-[5-(2,4-dichloro-phenyl)-3-(3-hydroxy-cyclohexylthiocarbamoyl)-2-methyl-pyrrol-1-yl]-phenyl ester
3-Fluoro-propane-1-sulfonic acid 4-[3-(3-amino-cyclohexylthiocarbamoyl)-5-(2,4-dichloro-phenyl)-2-methyl-pyrrol-1-yl]-phenyl ester
3-Fluoro-propane-1-sulfonic acid 4-[5-(2,4-dichloro-phenyl)-2-methyl-3-(5-trifluoromethyl-pyridin-2-ylthiocarbamoyl)-pyrrol-1-yl]-phenyl ester
3-Fluoro-propane-1-sulfonic acid 4-[5-(2,4-dichloro-phenyl)-3-(4,4-difluoro-cyclohexylthiocarbamoyl)-2-methyl-pyrrol-1-yl]-phenyl ester
5-(2,4-Dichloro-phenyl)-2-methyl-1-[4-(3,3,3-trifluoro-propoxy)-phenyl]-1H-pyrrole-3-carbothioic acid (4,4-difluoro-cyclohexyl)-amide
5-(2,4-Dichloro-phenyl)-1-[4-(3-fluoro-propoxy)-phenyl]-2-methyl-1H-pyrrole-3-carbothioic acid (4,4-difluoro-cyclohexyl)-amide
3,3,3-Trifluoro-propane-1-sulfonic acid 4-[5-(2,4-dichloro-phenyl)-3-(2-dimethylamino-cyclohexylthiocarbamoyl)-2-methyl-pyrrol-1-yl]-phenyl ester
3,3,3-Trifluoro-propane-1-sulfonic acid 4-[5-(2,4-dichloro-phenyl)-3-(2-hydroxy-cyclohexylthiocarbamoyl)-2-methyl-pyrrol-1-yl]-phenyl ester
3,3,3-Trifluoro-propane-1-sulfonic acid 4-[5-(2,4-dichloro-phenyl)-3-(4,4-difluoro-cyclohexylthiocarbamoyl)-2-methyl-pyrrol-1-yl]-phenyl ester
3,3,3-Trifluoro-propane-1-sulfonic acid 4-[4-(2,4-dichloro-phenyl)-2-(4,4-difluoro-cyclohexylthiocarbamoyl)-thiazol-5-yl]-phenyl ester
3,3,3-Trifluoro-propane-1-sulfonic acid 4-[4-(2,4-dichloro-phenyl)-2-(2-hydroxy-cyclohexylthiocarbamoyl)-thiazol-5-yl]-phenyl ester
3,3,3-Trifluoro-propane-1-sulfonic acid 4-[4-(2,4-dichloro-phenyl)-2-(2-dimethylamino-cyclohexylthiocarbamoyl)-thiazol-5-yl]-phenyl ester
3-Fluoro-propane-1-sulfonic acid 4-[4-(2,4-dichloro-phenyl)-2-(4,4-difluoro-cyclohexylthiocarbamoyl)-thiazol-5-yl]-phenyl ester
4-(2,4-Dichloro-phenyl)-5-[4-(3,3,3-trifluoro-propoxy)-phenyl]-thiazole-2-carbothioic acid (4,4-difluoro-cyclohexyl)-amide
5,6-Bis-(4-chloro-phenyl)-3-methoxymethyl-pyrazine-2-carbothioic acid (4,4-difluoro-cyclohexyl)-amide
5,6-Bis-(4-chloro-phenyl)-3-methoxymethyl-pyrazine-2-carbothioic acid (2-hydroxy-cyclohexyl)-amide
5,6-Bis-(4-chloro-phenyl)-3-methoxymethyl-pyrazine-2-carbothioic acid (2-dimethylamino-cyclohexyl)-amide
5,6-Bis-(4-chloro-phenyl)-3-methoxymethyl-pyrazine-2-carbothioic acid (3-hydroxy-cyclohexyl)-amide
5,6-Bis-(4-chloro-phenyl)-3-methoxymethyl-pyrazine-2-carbothioic acid (3-amino-cyclohexyl)-amide
6-(4-Chloro-phenyl)-3-methoxymethyl-5-[4-(3,3,3-trifluoro-propoxy)-phenyl]-pyrazine-2-carbothioic acid (4,4-difluoro-cyclohexyl)-amide
5,6-Bis-(4-chloro-phenyl)-3-methoxymethyl-pyrazine-2-carbothioic acid (1-hydroxymethyl-3-methyl-butyl)-amide
5,6-Bis-(4-chloro-phenyl)-N-(4,4-difluoro-cyclohexyl)-2-methoxymethyl-thionicotinamide
5,6-Bis-(4-chloro-phenyl)-N-(2-hydroxy-cyclohexyl)-2-methoxymethyl-thionicotinamide
5,6-Bis-(4-chloro-phenyl)-N-(2-dimethylamino-cyclohexyl)-2-methoxymethyl-thionicotinamide
5,6-Bis-(4-chloro-phenyl)-3-methoxymethyl-pyridine-2-carbothioic acid (3-hydroxy-cyclohexyl)-amide
5,6-Bis-(4-chloro-phenyl)-3-methoxymethyl-pyridine-2-carbothioic acid (3-amino-cyclohexyl)-amide
5-(4-Chloro-phenyl)-N-(4,4-difluoro-cyclohexyl)-2-methoxymethyl-6-[4-(3,3,3-trifluoro-propoxy)-phenyl]-thionicotinamide
5,6-Bis-(4-chloro-phenyl)-3-methoxymethyl-pyridine-2-carbothioic acid (1-hydroxymethyl-3-methyl-butyl)-amide
   and pharmaceutically acceptable salts thereof.

“Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of formula I which is sufficiently basic, for example an acid-addition salt with an inorganic or organic acid or a base-addition salt of a compound of Formula I for example a base-addition salt of a compound of Formula I which is sufficiently acidic with an inorganic (e.g. a metal hydroxide) or an organic base (e.g. an amine).

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, is anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rythms, and arrythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rythms, and arrythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma. In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rythms, and arrythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders(e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention may also be suitable for use in the regulation of bone mass and bone loss and therefore useful in the treatment of osteoporosis and other bone diseases.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)UD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar. For example the IC50 of Example 10 is 2.7 nM.

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example higher free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows. 10 μg of membranes prepared from cells stably transfected with the CB1 gene were suspended in 200 μl of 100mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples is were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter. Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)UD)) and the IC50 value determined as the concentration required to give half maximal inhibition of GTPγS binding under the conditions used. The compounds of the present invention are active at the CB1 receptor (IC50<1 micromolar). Most preferred compounds have IC50<200 nanomolar.

The compounds of the invention are believed to be selective CB1 antagonists.

### Methods of Preparation

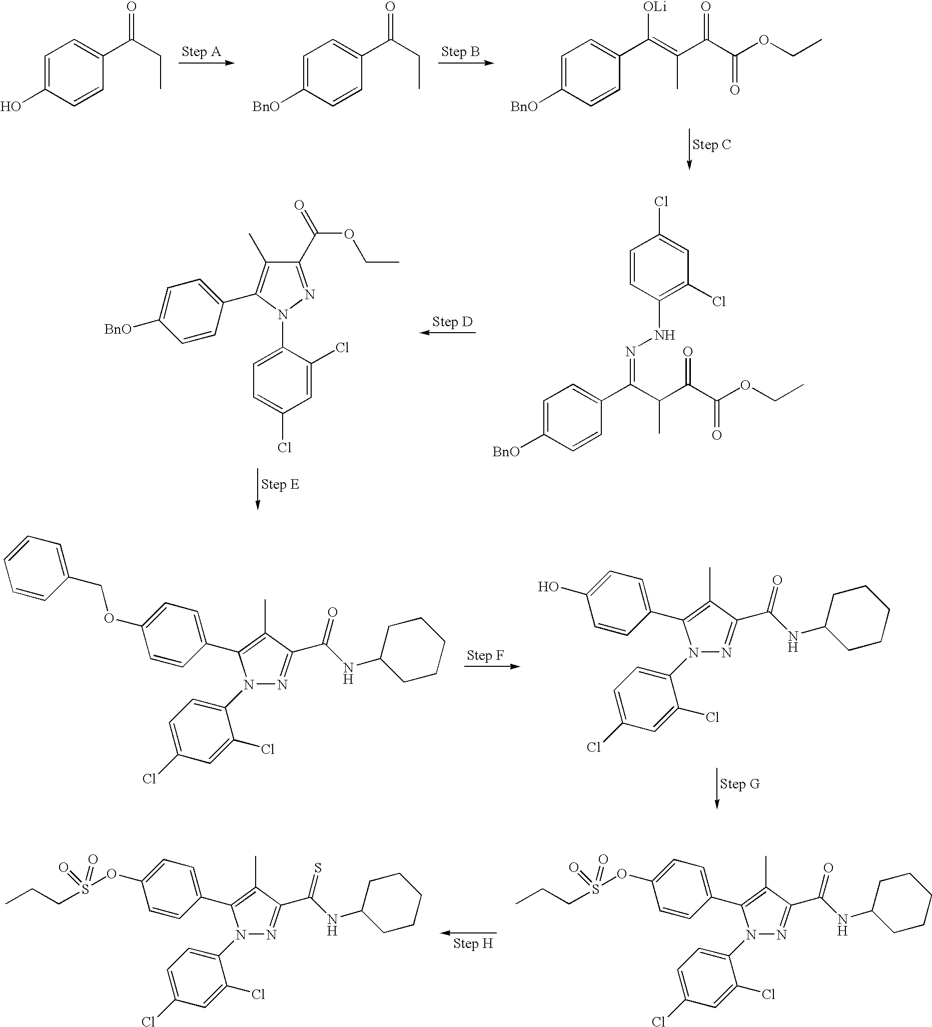

The present invention provides a process for the preparation of a compound of formula I comprising reacting a compound of formula II

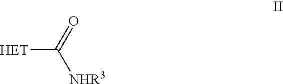

in which Het and R³ are as previously defined with Lawesson's reagent in the presence of a diluent for example toluene at a temperature in the range of 50° C. to 250° C. preferably 100° C. to 200° C.

### EXAMPLES

### Abbreviations

AcOH acetic acid
aq aqueous
DCM dichloromethane
DMF dimethylformamide
DEA diethylamine
DEAD diethyl azodicarboxylate

### DIEA N,N-diisopropylethylamine

DMAP 4-dimethylaminopyridine
EtOAc ethyl acetate
Et₃N triethylamine

### Ex or EX Example

LiHMDS lithium hexamethyldisilazide
NH₄Ac ammonium acetate
MeOH methanol
MeCN acetonitrile
rt or RT room temperature
TEA triethylamine
THF tetrahydrofuran
t triplet
s singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at 1H frequencies of 300 and 500 MHz respectively. Chemical shifts are given in ppm with CDCl₃ as internal standard. is CDCl₃ is used as the solvent for NMR unless otherwise stated. Purification was performed on a semipreparative HPLC (High Performance Liquid Chromatography) with a mass triggered fraction collector, Shimadzu QP 8000 single quadrupole mass spectrometer equipped with 19×100 mm C8 column. The mobile phase used was, if nothing else is stated, acetonitrile and buffer (0.1 M ammonium acetate:acetonitrile 95:5).

For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

Typical HPLC-parameters for purity analysis:

### HPLC-system: Agilent 1100

### Column: Zorbax Eclipse XDB-C8 150×4.6 mm

Time of analysis: 15 min
Flow: 1.5 ml/min
Mobilphase: A: water, 5% MeOH
B: MeOH

### Temperature: 40° C.

### Detector: Uv 240 nm

### Example 1

### Step A 1-(4-Benzyloxyphenyl)-propan-1-one

To a solution of 4-hydroxypropiophenone (50 g, 0.3329 mol) in dry acetone (500 ml) was added benzyl bromide (56.94 g, 0.333 mol) followed by anhydrous K₂CO₃ (91.8 g, 0.665 mol). Reaction mixture was boiled under reflux for 18 h, cooled to RT, filtered and filtrate was concentrated to yield 4-benzyloxy propiophenone (75 g, 93%) as a white solid.

### Step B Lithium 1-(4-benzyloxyphenyl)-3-ethoxycarbonyl-2-methyl-3-oxo-propen-1-olate

To a solution of 4-benzyloxypropiophenone (50 g, 0.2083 mol) in dry THF (500 ml) at 0° C. was added LiHMDS (1M solution in THF, 208.3 ml) dropwise over a period of 1 h under a nitrogen atmosphere. The reaction mixture was stirred at 0° C. for 1 hr. Diethyl oxalate was added (33.49 g, 0.2296 mol) dropwise. The reaction mixture was allowed to warm to RT and stirred at RT for 16 hrs under a nitrogen atmosphere. The reaction mixture was concentrated in the rotavap at RT. Dry diethyl ether (1 L) was added to the residue and the solid was collected by filtration, washed with dry ether, and dried under vacuum to yield lithium salt of the diketoester (50 g) as yellow solid.

### Step C 4-(4-Benzyloxyphenyl)-4-[(2,4-dichlorophenyl)-hydrazono]-3-methyl-2-oxo-butyric acid ethyl ester

A mixture of lithium salt from step C (50 g, 0.1461 mol) and 2,4-dichlorophenylhydrazine hydrochloride (34.33 g, 0.1608 mol) in ethanol (500 ml) was stirred at RT under a nitrogen atmosphere for 18 hrs. The precipitate was filtered, washed with dry ether and dried under vacuum to yield hydrazone intermediate (35 g).

### Step D Ethyl 5-[4-(benzyloxy)phenyl]-1-(2,4-dichlorophenyl)-4-methyl-1H-pyrazole-3-carboxylate

Hydrazone intermediate (35 g) was dissolved in acetic acid (250 ml) and heated under reflux for 18 hrs. Reaction mixture was poured into cold water (2 L) and extracted with ethyl acetate (2×500 ml). Combined organic layer was washed with water, sat. NaHCO₃ and brine, dried over Na₂SO₄, concentrated and purified by column chromatography over silica gel using 20% ethyl acetate in petroleum ether as eluent to yield the title compound as yellow solid.

### Step E 5-[4-(benzyloxy)phenyl]-N-cyclohexyl-1-(2,4-dichlorophenyl)-4-methyl-1H-pyrazole-3-carboxamide

Cyclohexylamine hydrochloride (1.63 g, 12.0 mmol) was suspended in anhydrous toluene (12 mL) under an atmosphere of Argon. Upon cooling to 0° C. trimethylaluminum (6.0 mL, 2.0 M in toluene, 12 mmol) was added dropwise at such a rate as to keep the evolution of methane under control. The obtained mixture was stirred at 0° C. for 1 h and then at ambient temperature for additional two hours before use. It was assumed that the formation of the desired aluminium amide was quantitative and the concentration thereof was thus calculated to be ca 0.67 M [c=12.0/(12+6.0)]. At that point ethyl 5-[4-(benzyloxy)phenyl]-1-(2,4-dichlorophenyl)-4-methyl-1H-pyrazole-3-carboxylate (1.93 g, 4.00 mmol) was added in one portion to the prepared aluminium amide and the resulting orange-coloured suspension was heated at 50° C. overnight. Upon cooling to 0° C. the reaction was quenched by the dropwise addition of HCl (aq., 2 M). The addition continued until no further evolution of gas could be observed and at that point the ice bath was removed and the mixture was allowed to stir at ambient temperature for an additional hour. The obtained mixture was transferred to a separation funnel with the aid of CH₂Cl₂ (100 mL). H₂O (100 mL) was added and the pH of the aqueous phase was adjusted to 4-6. The organic phase was separated and the aqueous phase was extracted further with CH₂Cl₂ (4×30 mL). The collected organic phases were combined and washed with brine before drying over MgSO₄. Upon evaporation of the solvents the obtained residue was purified by column chromatography (silica gel, EtOAc-CH₂Cl₂, 0-5%) to yield the 5-[4-(benzyloxy)phenyl]-N-cyclohexyl-1-(2,4-dichlorophenyl)-4-methyl-1H-pyrazole-3-carboxamide (2.04 g, 3.83 mmol, 96%) as a yellow solid.

1H NMR (500 MHz, CDCl3) δ 7.50-7.24 (m, 8H), 7.06 (d, 2H, J=8.8 Hz), 6.92 (d, 2H, J=8.8 Hz), 6.85 (br d, 1H, J=8.4 Hz), 5.05 (s, 2H), 4.05-3.90 (m, 1H), 2.39 (s, 3H), 2.10-1.98 (m, 2H), 1.82-1.72 (m, 2H), 1.70-1.60 (m, 1H), 1.50-1.36 (m, 2H), 1.34-1.14 (m, 3H).

### Step F N-cyclohexyl-1-(2,4-dichlorophenyl)-5-(4-hydroxyphenyl)-4-methyl-1H-pyrazole-3-carboxamide

The 5-[4-(benzyloxy)phenyl]-N-cyclohexyl-1-(2,4-dichlorophenyl)-4-methyl-1H-pyrazole-3-carboxamide (2.030 g, 3.80 mmol) was dissolved in anhydrous CH₂Cl₂ (38 mL) under an atmosphere of Argon (Ar). Dimethyl sulfide (5.6 mL, 76 mmol) was added in one portion and, finally, boron trifluoride-diethyl etherate (4.8 mL, 38 mmol) was added during 5 min. After stirring the reaction mixture at ambient temperature for 4 days it was transferred to a separation funnel with the aid of CH₂Cl₂ (100 mL). NaHCO3 (aq., sat., 150 mL) was added. The organic phase was separated and the aqueous phase was extracted further with CH₂Cl₂ (3×50 mL). The collected organic phases were dried over MgSO4. Upon evaporation of the solvents the obtained residue was purified by column chromatography (silica gel, EtOAc-CH₂Cl₂, 0-15%) to yield the N-cyclohexyl-1-(2,4-dichlorophenyl)-5-(4-hydroxyphenyl)-4-methyl-1H-pyrazole-3-carboxamide (1.637 g, 3.68 mmol, 97%) as an off-white powder.

1H NMR (500 MHz, CDCl3) δ7.43 (s, 1H), 7.31-7.26 (m, 2H), 6.99 (d, 2H, J=8.7 Hz), is 6.90 (br d, 1H, J=8.3 Hz), 6.82 (d, 2H, J=8.7 Hz), 6.19 (br s, 1H), 4.02-3.89 (m, 1H), 2.37 (s, 3H), 2.08-1.96 (m, 2H), 1.82-1.70 (m, 2H), 1.70-1.58 (m, 1H), 1.48-1.34 (m, 2H), 1.34-1.12 (m, 3H).

### Step G 4-[3-[(cyclohexylamino)carbonyl]-1-(2,4-dichlorophenyl)-4-methyl-1H-pyrazol-5-yl]phenyl propane-1-sulfonate

The N-cyclohexyl-1-(2,4-dichlorophenyl)-5-(4-hydroxyphenyl)-4-methyl-1H-pyrazole-3-carboxamide (0.910 g, 2.05 mmol) was suspended in anhydrous CH₂Cl₂ (20 mL) under an atmosphere of Argon and triethylamine (430 μL, 0.31 g, 3.07 mmol) was added in one portion. Upon cooling to 0° C. propanesulfonyl chloride (270 μL, 0.35 g, 2.5 mmol) was added dropwise during 5 min. When the addition had been completed the ice bath was removed and the reaction mixture was allowed to reach ambient temperature. After stirring overnight the mixture was transferred to a separation funnel with the aid of CH₂Cl₂ (130 mL). The organic phase was then washed with H₂O (40 mL) and brine, and finally dried over MgSO₄. Upon evaporation of the solvents the obtained residue was purified by 30 column chromatography (silica gel, EtOAc- CH₂Cl₂, 0-8%) to yield the 4-[3-[(cyclohexylamino)carbonyl]-1-(2,4-dichlorophenyl)-4-methyl-1H-pyrazol-5-yl]phenyl propane-1-sulfonate (1.117 g, 2.03 mmol, 99%) as a pinkish solid.

¹H NMR (500 MHz, CDCl₃) δ 7.44 (s, 1H), 7.36-7.28 (m, 2H), 7.24 (d, 2H, J=8.8 Hz), 7.18 (d, 2H, J=8.8 Hz), 6.84 (br d, 1H, J=8.4 Hz), 4.04-3.90 (m, 1H), 3.30-3.20 (m, 2H), 2.40 (s, 3H), 2.10-1.96 (m, 4H), 1.84-1.72 (m, 2H), 1.72-1.60 (m, 1H), 1.50-1.36 (m, 2H), 1.34-1.16 (m, 3H), 1.14 (t, 3H, J=7.4 Hz).

### Step H 4-[3-[(cyclohexylamino)carbonothioyl]-1-(2,4-dichlorophenyl)-4-methyl-1H-pyrazol-5-yl]phenyl propane-1-sulfonate

The 4-[3-[(cyclohexylamino)carbonyl]-1-(2,4-dichlorophenyl)-4-methyl-1H-pyrazol-5-yl]phenyl propane-1-sulfonate (0.300 g, 0.545 mmol) and Lawesson's reagent (0.441 g, 1.09 mmol) were suspended in toluene (22 mL) and heated in a microwave oven (single node heating) at 150° C. for 10 min. Upon cooling the solvents were evaporated and the obtained residue was purified by column chromatography (silica gel, CH₂Cl₂) to yield the 4-[3-[(cyclohexylamino)carbonothioyl]-1-(2,4-dichlorophenyl)-4-methyl-1H-pyrazol-5-yl]phenyl propane-1-sulfonate (0.307 g, 0.542 mmol, 99%) as a yellow viscous oil, which solidified upon standing.

¹H NMR (500 MHz, CDCl₃) [symbol] 8.62 (br d, 1H, J=8.2 Hz), 7.44 (s, 1H), 7.34-7.22 (m, 4H), 7.19 (d, 2H, J=8.8 Hz), 4.66-4.52 (m, 1H), 3.32-3.20 (m, 2H), 2.49 (s, 3H), 2.22-2.16 (m, 2H), 2.10-1.96 (m, 2H), 1.86-1.74 (m, 2H), 1.74-1.64 (m, 1H), 1.56-1.42 (m, 2H), 1.42-1.31 (m, 2H), 1.31-1.18 (m, 1H), 1.14 (t, 3H, J=7.4 Hz).

HRMS Calcd for [C26H29Cl2N3O3S2+H]+: 566.1106. Found: 566.1130.

The compounds listed below were prepared by methods analogous to those described above with Lawesson's reagent heated in a microwave oven or with conventional heating. The starting carboxamides may be obtained as described in EP 656354 (pyrazoles), WO03/27076 (imidazoles), pyrroles (WO2004/058249), thiazoles (WO2004/058249), pyrazines (WO03/051851) and pyridines by methods analogous to those in WO03/051851.

It will be appreciated by those skilled in the art that certain groups that might interfere in this reaction may be protected before the reaction with Lawesson's reagent and then deprotected after the reaction by methods known to those skilled in the art. For example a hydroxymethyl substituent may be protected as methoxymethyl or benzyloxymethyl and then regenerated by methods known to those skilled in the art after reaction with Lawesson's reagent.

### Example 2

5-(4-Chlorophenyl)-1-(2,4-dichlorophenyl)-4-methyl-1H-pyrazole-3-carbothioic acid (4,4-difluorocyclohexyl)amide.

### Example 3

5-(2,4-Dichlorophenyl)-1-(4-methoxyphenyl)-2-methyl-1H-pyrrole-3-carbothioic acid(4,4-difluorocyclohexyl)amide.

### Example 4

5,6-Bis-(4-chlorophenyl)-3-methoxymethyl-pyrazine-2-carbothioic acid (4,4-difluoro-cyclohexyl)amide.

### Example 5

5,6-Bis-(4-chlorophenyl)pyrazine-2-carbothioic acid cyclohexylamide.

### Example 6

### 5,6-Bis-(4-chlorophenyl)pyrazine-2-carbothioic acid (4,4-difluorocyclohexyl)amide

The compounds listed below are prepared by methods analogous to those described above with Lawesson's reagent heated in a microwave oven or with conventional heating.

### Pyrazoles:

1) 3,3,3-Trifluoro-propane-1-sulfonic acid 4-[2-(2,4-dichloro-phenyl)-5-(4,4-difluorocyclohexylthiocarbamoyl)-4-methyl-2H-pyrazol-3-yl]-phenylester
2) 3,3,3-Trifluoro-propane-1-sulfonic acid 4-[2-(2,4-dichloro-phenyl)-5-(2-hydroxycyclohexylthiocarbamoyl)-4-methyl-2H-pyrazol-3-yl]-phenyl ester
3) 3,3,3-Trifluoro-propane-1-sulfonic acid 4-[2-(2,4-dichloro-phenyl)-5-(2-dimethylaminocyclohexylthiocarbamoyl)-4-methyl-2H-pyrazol-3-yl]-phenyl ester
4) 3-Fluoro-propane-1-sulfonic acid 4-[2-(2,4-dichloro-phenyl)-5-(4,4-difluorocyclohexylthiocarbamoyl)-4-methyl-2H-pyrazol-3-yl]-phenyl ester
5) 1-(2,4-Dichloro-phenyl)-4-methyl-5-[4-(3,3,3-trifluoro-propoxy)-phenyl]-1H-pyrazole-3-carbothioic acid (4,4-difluoro-cyclohexyl)amide
6) 1-(2,4-Dichloro-phenyl)-5-[4-(3-fluoropropoxy)-phenyl]-4-methyl-1H-pyrazole-3-carbothioic acid (4,4-difluoro-cyclohexyl)amide
7) 3-Fluoro-propane-1-sulfonic acid 4-[2-(2,4-dichloro-phenyl)-5-(3-hydroxy-cyclohexylthiocarbamoyl)-4-methyl-2H-pyrazol-3-yl]phenyl ester
8) 3-Fluoro-propane-1-sulfonic acid 4-[5-(3-amino-cyclohexylthiocarbamoyl)-2-(2,4-dichloro-phenyl)-4-methyl-2H-pyrazol-3-yl]-phenyl ester
9) 3-Fluoro-propane-1-sulfonic acid 4-[2-(2,4-dichlorophenyl)-4-methyl-5-(5-trifluoromethyl-pyridin-2-ylthiocarbamoyl)-2H-pyrazol-3-yl]phenyl ester

### Imidazoles

1) 3,3,3-Trifluoro-propane-1-sulfonic acid 4-[2-(2,4-dichloro-phenyl)-4-(4,4-difluorocyclohexylthiocarbamoyl)-5-methyl-imidazol-1-yl]-phenyl ester
2) 3,3,3-Trifluoro-propane-1-sulfonic acid 4-[2-(2,4-dichloro-phenyl)-4-(2-hydroxy-cyclohexylthiocarbamoyl)-5-methyl-imidazol-1-yl]-phenyl ester
3) 3,3,3-Trifluoro-propane-1-sulfonic acid 4-[2-(2,4-dichloro-phenyl)-4-(2-dimethylamino-cyclohexylthiocarbamoyl)-5-methyl-imidazol-1-yl]-phenyl ester
4) 3-Fluoro-propane-1-sulfonic acid 4-[2-(2,4-dichloro-phenyl)-4-(4,4-difluoro-cyclohexylthiocarbamoyl)-5-methyl-imidazol-1-yl]-phenyl ester
5) 2-(2,4-Dichloro-phenyl)-5-methyl-1-[4-(3,3,3-trifluoro-propoxy)-phenyl]-1H-imidazole-4-carbothioic acid (4,4-difluoro-cyclohexyl)-amide
6) 2-(2,4-Dichloro-phenyl)-1-[4-(3-fluoro-propoxy)-phenyl]-5-methyl-1H-imidazole-4-carbothioic acid (4,4-difluoro-cyclohexyl)-amide
7) 3-Fluoro-propane-1-sulfonic acid 4-[2-(2,4-dichloro-phenyl)-4-(3-hydroxy-cyclohexylthiocarbamoyl)-5-methyl-imidazol-1-yl]-phenyl ester
8) 3-Fluoro-propane-1-sulfonic acid 4-[4-(3-amino-cyclohexylthiocarbamoyl)-2-(2,4-dichloro-phenyl)-5-methyl-imidazol-1-yl]-phenyl ester
9) 3-Fluoro-propane-1-sulfonic acid 4-[2-(2,4-dichloro-phenyl)-5-methyl-4-(5-trifluoromethyl-pyridin-2-ylthiocarbamoyl)-imidazol-1-yl]-phenyl ester

### Pyrroles

1) 3-Fluoro-propane-1-sulfonic acid 4-[5-(2,4-dichloro-phenyl)-3-(3-hydroxy-cyclohexylthiocarbamoyl)-2-methyl-pyrrol-1-yl]-phenyl ester
2) 3-Fluoro-propane-1-sulfonic acid 4-[3-(3-amino-cyclohexylthiocarbamoyl)-5-(2,4-dichloro-phenyl)-2-methyl-pyrrol-1-yl]-phenyl ester
3) 3-Fluoro-propane-1-sulfonic acid 4-[5-(2,4-dichloro-phenyl)-2-methyl-3-(5-trifluoromethyl-pyridin-2-ylthiocarbamoyl)-pyrrol-1-yl]-phenyl ester
4) 3-Fluoro-propane-1-sulfonic acid 4-[5-(2,4-dichloro-phenyl)-3-(4,4-difluoro-cyclohexylthiocarbamoyl)-2-methyl-pyrrol-1-yl]-phenyl ester
5) 5-(2,4-Dichloro-phenyl)-2-methyl-1-[4-(3,3,3-trifluoro-propoxy)-phenyl]-1H-pyrrole-3-carbothioic acid (4,4-difluoro-cyclohexyl)-amide
6) 5-(2,4-Dichloro-phenyl)-1-[4-(3-fluoro-propoxy)-phenyl]-2-methyl-1H-pyrrole-3-carbothioic acid (4,4-difluoro-cyclohexyl)-amide
7) 3,3,3-Trifluoro-propane-1-sulfonic acid 4-[5-(2,4-dichloro-phenyl)-3-(2-dimethylamino-cyclohexylthiocarbamoyl)-2-methyl-pyrrol-1-yl]-phenyl ester
8) 3,3,3-Trifluoro-propane-1-sulfonic acid 4-[5-(2,4-dichloro-phenyl)-3-(2-hydroxy-cyclohexylthiocarbamoyl)-2-methyl-pyrrol-1-yl]-phenyl ester
9) 3,3,3-Trifluoro-propane-1-sulfonic acid 4-[5-(2,4-dichloro-phenyl)-3-(4,4-difluoro-cyclohexylthiocarbamoyl)-2-methyl-pyrrol-1-yl]-phenyl ester

### Thiazoles

1) 3,3,3-Trifluoro-propane-1-sulfonic acid 4-[4-(2,4-dichloro-phenyl)-2-(4,4-difluoro-cyclohexylthiocarbamoyl)-thiazol-5-yl]-phenyl ester
2) 3,3,3-Trifluoro-propane-1-sulfonic acid 4-[4-(2,4-dichloro-phenyl)-2-(2-hydroxy-cyclohexylthiocarbamoyl)-thiazol-5-yl]-phenyl ester
3) 3,3,3-Trifluoro-propane-1-sulfonic acid 4-[4-(2,4-dichloro-phenyl)-2-(2-dimethylamino-cyclohexylthiocarbamoyl)-thiazol-5-yl]-phenyl ester
4) 3-Fluoro-propane-1-sulfonic acid 4-[4-(2,4-dichloro-phenyl)-2-(4,4-difluoro-cyclohexylthiocarbamoyl)-thiazol-5-yl]-phenyl ester
5) 4-(2,4-Dichloro-phenyl)-5-[4-(3,3,3-trifluoro-propoxy)-phenyl]-thiazole-2-carbothioic acid (4,4-difluoro-cyclohexyl)-amide

### Pyrazines

1) 5,6-Bis-(4-chloro-phenyl)-3-methoxymethyl-pyrazine-2-carbothioic acid (1-hydroxymethyl-3-methyl-butyl)amide
2) 5,6-Bis-(4-chloro-phenyl)-3-methoxymethyl-pyrazine-2-carbothioic acid (2-hydroxy-cyclohexyl)amide
3) 5,6-Bis-(4-chloro-phenyl)-3-methoxymethyl-pyrazine-2-carbothioic acid (2-dimethylamino-cyclohexyl)amide
4) 5,6-Bis-(4-chloro-phenyl)-3-methoxymethyl-pyrazine-2-carbothioic acid (3-hydroxy-cyclohexyl)amide
5) 5,6-Bis-(4-chloro-phenyl)-3-methoxymethyl-pyrazine-2-carbothioic acid (3-amino-cyclohexyl)amide
6) 6-(4-Chloro-phenyl)-3-methoxymethyl-5-[4-(3,3,3-trifluoro-propoxy)-phenyl]-pyrazine-2-carbothioic acid (4,4-difluoro-cyclohexyl)amide

### Pyridines

1) 5,6-Bis-(4-chloro-phenyl)-N-(4,4-difluoro-cyclohexyl)-2-methoxymethyl-thionicotinamide
2) 5,6-Bis-(4-chloro-phenyl)-N-(2-hydroxy-cyclohexyl)-2-methoxymethyl-thionicotinamide
3) 5,6-Bis-(4-chloro-phenyl)-N-(2-dimethylamino-cyclohexyl)-2-methoxymethyl-thionicotinamide
4) 5,6-Bis-(4-chloro-phenyl)-3-methoxymethylpyridine-2-carbothioic acid (3-hydroxy-cyclohexyl)amide
5) 5,6-Bis-(4-chloro-phenyl)-3-methoxymethylpyridine-2-carbothioic acid (3-amino-cyclohexyl)amide
6) 5-(4-Chloro-phenyl)-N-(4,4-difluorocyclohexyl)-2-methoxymethyl-6-[4-(3,3,3-trifluoro-propoxy)-phenyl]-thionicotinamide
7) 5,6-Bis-(4-chloro-phenyl)-3-methoxymethylpyridine-2-carbothioic acid (1-hydroxymethyl-3-methylbutyl)amide

1. A compound of formula (I) 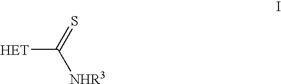
or a pharmaceutically acceptable salt thereof wherein HET represents a group selected from:
a) a group of formula A 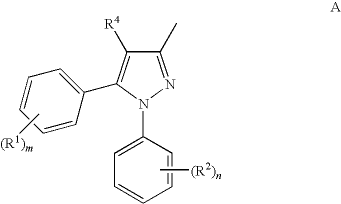
b) a group of formula B 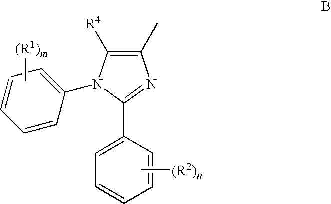
c) a group of formula C 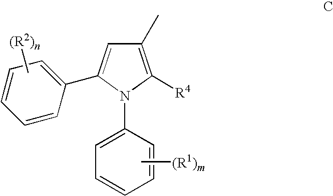
d) a group of formula D 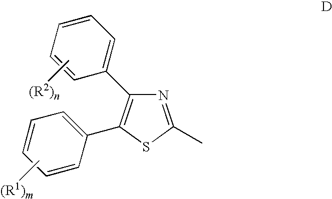
e) a group of formula E 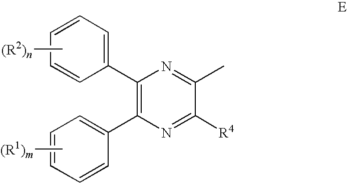
f) a group of formula F 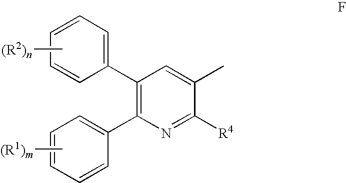
and
g) a group of formula G 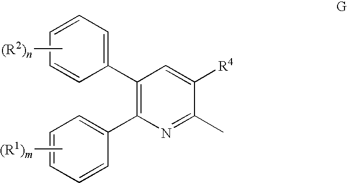
wherein
R¹ represents a) a C₁₋₁₀alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O in which R⁵ represents a C₁₋₁₀alkyl group optionally substituted by one or more fluoro or d) halo or e) a C₁₋₄alkyl group;
m is 0, 1, 2 or 3;
R² represents a C₁₋₃alkyl group, a C₁₋₃alkoxy group, hydroxy, nitro, cyano or halo;
n is 0, 1, 2 or 3;
R³ represents a) cyclohexyl optionally substituted by one or more of the following: hydroxy, fluoro, amino, mono or di C₁₋₃alkylamino b) piperidino optionally substituted by one or more hydroxy c) phenyl optionally substituted by one or more of the following: hydroxy, halo or a C₁₋₄alkyl group d) pyridyl optionally substituted by a C₁₋₄alkyl group or e) a C₄₋₉alkyl group; and
R⁴ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more hydroxy, fluoro, amino, monoC₁₋₄alkylamino, diC₁₋₄alkylamino or cyano.

2. The compound according to claim 1 wherein HET represents a group of formula A and R¹, R², R³, R⁴, n and m are as defined in claim 1.

3. The compound according to claim 1 wherein HET represents a group of formula B and R¹, R², R³, R⁴, n and m are as defined in claim 1.

4. The compound according to claim 1 wherein HET represents a group of formula C and R¹, R², R³, R⁴, n and m are as defined in claim 1.

5. The compound according to claim 1 wherein HET represents a group of formula D and R¹, R², R³, R⁴, n and m are as defined in claim 1.

6. The compound according to claim 1 wherein HET represents a group of formula E and R¹, R², R³, R⁴, n and m are as defined in claim 1.

7. The compound according to claim 1 wherein HET represents a group of formula F and R¹, R², R³, R⁴, n and m are as defined in claim 1.

8. The compound according to claim 1 wherein HET represents a group of formula G and R¹, R², R³, R⁴, n and m are as defined in claim 1.

9. The compound according to claim 1 selected from:
3,3,3-Trifluoro-propane-1-sulfonic acid-4-[2-(2,4-dichloro-phenyl)-5-(4,4-difluorocyclohexylthiocarbamoyl)-4-methyl-2H-pyrazol-3-yl]-phenylester
3,3,3-Trifluoro-propane-1-sulfonic acid 4-[2-(2,4-dichloro-phenyl)-5-(2-hydroxycyclohexylthiocarbamoyl)-4-methyl-2H-pyrazol-3-yl]-phenyl ester
3,3,3-Trifluoro-propane-1-sulfonic acid 4-[2-(2,4-dichloro-phenyl)-5-(2-dimethylaminocyclohexylthiocarbamoyl)-4-methyl-2H-pyrazol-3-yl]-phenyl ester
3-Fluoro-propane-1-sulfonic acid 4-[2-(2,4-dichloro-phenyl)-5-(4,4-difluorocyclohexylthiocarbamoyl)-4-methyl-2H-pyrazol-3-yl]-phenyl ester
1-(2,4-Dichloro-phenyl)-4-methyl-5-[4-(3,3,3-trifluoro-propoxy)-phenyl]-1H-pyrazole-3-carbothioic acid (4,4-difluoro-cyclohexyl)-amide
1-(2,4-Dichloro-phenyl)-5-[4-(3-fluoro-propoxy)-phenyl]-4-methyl-1H-pyrazole-3-carbothioic acid (4,4-difluoro-cyclohexyl)-amide
3-Fluoro-propane-1-sulfonic acid 4-[2-(2,4-dichloro-phenyl)-5-(3-hydroxy-cyclohexylthiocarbamoyl)-4-methyl-2H-pyrazol-3-yl]-phenyl ester
3-Fluoro-propane-1-sulfonic acid 4-[5-(3-amino-cyclohexylthiocarbamoyl)-2-(2,4-dichloro-phenyl)-4-methyl-2H-pyrazol-3-yl]-phenyl ester
3-Fluoro-propane-1-sulfonic acid 4-[2-(2,4-dichloro-phenyl)-4-methyl-5-(5-trifluoromethyl-pyridin-2-ylthiocarbamoyl)-2H-pyrazol-3-yl]-phenyl ester
3,3,3-Trifluoro-propane-1-sulfonic acid 4-[2-(2,4-dichloro-phenyl)-4-(4,4-difluorocyclohexylthiocarbamoyl)-5-methyl-imidazol-1-yl]-phenyl ester
3,3,3-Trifluoro-propane-1-sulfonic acid 4-[2-(2,4-dichloro-phenyl)-4-(2-hydroxy-cyclohexylthiocarbamoyl)-5-methyl-imidazol-1-yl]-phenyl ester
3,3,3-Trifluoro-propane-1-sulfonic acid 4-[2-(2,4-dichloro-phenyl)-4-(2-dimethylamino-cyclohexylthiocarbamoyl)-5-methyl-imidazol-1-yl]-phenyl ester
3-Fluoro-propane-1-sulfonic acid 4-[2-(2,4-dichloro-phenyl)-4-(4,4-difluoro-cyclohexylthiocarbamoyl)-5-methyl-imidazol-1-yl]-phenyl ester
2-(2,4-Dichloro-phenyl)-5-methyl-1-[4-(3,3,3-trifluoro-propoxy)-phenyl]-1H-imidazole-4-carbothioic acid (4,4-difluoro-cyclohexyl)-amide
2-(2,4-Dichloro-phenyl)-1-[4-(3-fluoro-propoxy)-phenyl]-5-methyl-1H-imidazole-4-carbothioic acid (4,4-difluoro-cyclohexyl)-amide
3-Fluoro-propane-1-sulfonic acid 4-[2-(2,4-dichloro-phenyl)-4-(3-hydroxy-cyclohexylthiocarbamoyl)-5-methyl-imidazol-1-yl]-phenyl ester
3-Fluoro-propane-1-sulfonic acid 4-[4-(3-amino-cyclohexylthiocarbamoyl)-2-(2,4-dichloro-phenyl)-5-methyl-imidazol-1-yl]-phenyl ester
3-Fluoro-propane-1-sulfonic acid 4-[2-(2,4-dichloro-phenyl)-5-methyl-4-(5-trifluoromethyl-pyridin-2-ylthiocarbamoyl)-imidazol-1-yl]-phenyl ester
3-Fluoro-propane-1-sulfonic acid 4-[5-(2,4-dichloro-phenyl)-3-(3-hydroxy-cyclohexylthiocarbamoyl)-2-methyl-pyrrol-1-yl]-phenyl ester
3-Fluoro-propane-1-sulfonic acid 4-[3-(3-amino-cyclohexylthiocarbamoyl)-5-(2,4-dichloro-phenyl)-2-methyl-pyrrol-1-yl]-phenyl ester
3-Fluoro-propane-1-sulfonic acid 4-[5-(2,4-dichloro-phenyl)-2-methyl-3-(5-trifluoromethyl-pyridin-2-ylthiocarbamoyl)-pyrrol-1-yl]-phenyl ester
3-Fluoro-propane-1-sulfonic acid 4-[5-(2,4-dichloro-phenyl)-3-(4,4-difluoro-cyclohexylthiocarbamoyl)-2-methyl-pyrrol-1-yl]-phenyl ester
5-(2,4-Dichloro-phenyl)-2-methyl-1-[4-(3,3,3-trifluoro-propoxy)-phenyl]-1H-pyrrole-3-carbothioic acid (4,4-difluoro-cyclohexyl)-amide
5-(2,4-Dichloro-phenyl)-1-[4-(3-fluoro-propoxy)-phenyl]-2-methyl-1H-pyrrole-3-carbothioic acid (4,4-difluoro-cyclohexyl)-amide
3,3,3-Trifluoro-propane-1-sulfonic acid 4-[5-(2,4-dichloro-phenyl)-3-(2-dimethylamino-cyclohexylthiocarbamoyl)-2-methyl-pyrrol-1-yl]-phenyl ester
3,3,3-Trifluoro-propane-1-sulfonic acid 4-[5-(2,4-dichloro-phenyl)-3-(2-hydroxy-cyclohexylthiocarbamoyl)-2-methyl-pyrrol-1-yl]-phenyl ester
3,3,3-Trifluoro-propane-1-sulfonic acid 4-[5-(2,4-dichloro-phenyl)-3-(4,4-difluoro-cyclohexylthiocarbamoyl)-2-methyl-pyrrol-1-yl]-phenyl ester
3,3,3-Trifluoro-propane-1-sulfonic acid 4-[4-(2,4-dichloro-phenyl)-2-(4,4-difluoro-cyclohexylthiocarbamoyl)-thiazol-5-yl]-phenyl ester
3,3,3-Trifluoro-propane-1-sulfonic acid 4-[4-(2,4-dichloro-phenyl)-2-(2-hydroxy-cyclohexylthiocarbamoyl)-thiazol-5-yl]-phenyl ester
3,3,3-Trifluoro-propane-1-sulfonic acid 4-[4-(2,4-dichloro-phenyl)-2-(2-dimethylamino-cyclohexylthiocarbamoyl)-thiazol-5-yl]-phenyl ester
3-Fluoro-propane-1-sulfonic acid 4-[4-(2,4-dichloro-phenyl)-2-(4,4-difluoro-cyclohexylthiocarbamoyl)-thiazol-5-yl]-phenyl ester
4-(2,4-Dichloro-phenyl)-5-[4-(3,3,3-trifluoro-propoxy)-phenyl]-thiazole-2-carbothioic acid (4,4-difluoro-cyclohexyl)-amide
5,6-Bis-(4-chloro-phenyl)-3-methoxymethyl-pyrazine-2-carbothioic acid (4,4-difluoro-cyclohexyl)-amide
5,6-Bis-(4-chloro-phenyl)-3-methoxymethyl-pyrazine-2-carbothioic acid (2-hydroxy-cyclohexyl)-amide
5,6-Bis-(4-chloro-phenyl)-3-methoxymethyl-pyrazine-2-carbothioic acid (2-dimethylamino-cyclohexyl)-amide
5,6-Bis-(4-chloro-phenyl)-3-methoxymethyl-pyrazine-2-carbothioic acid (3-hydroxy-cyclohexyl)-amide
5,6-Bis-(4-chloro-phenyl)-3-methoxymethyl-pyrazine-2-carbothioic acid (3-amino-cyclohexyl)-amide
6-(4-Chloro-phenyl)-3-methoxymethyl-5-[4-(3,3,3-trifluoro-propoxy)-phenyl]-pyrazine-2-carbothioic acid (4,4-difluoro-cyclohexyl)-amide
5,6-Bis-(4-chloro-phenyl)-3-methoxymethyl-pyrazine-2-carbothioic acid (1-hydroxymethyl-3-methyl-butyl)-amide
5,6-Bis-(4-chloro-phenyl)-N-(4,4-difluoro-cyclohexyl)-2-methoxymethyl-thionicotinamide
5,6-Bis-(4-chloro-phenyl)-N-(2-hydroxy-cyclohexyl)-2-methoxymethyl-thionicotinamide
5,6-Bis-(4-chloro-phenyl)-N-(2-dimethylamino-cyclohexyl)-2-methoxymethyl-thionicotinamide
5,6-Bis-(4-chloro-phenyl)-3-methoxymethyl-pyridine-2-carbothioic acid (3-hydroxy-cyclohexyl)-amide
5,6-Bis-(4-chloro-phenyl)-3-methoxymethyl-pyridine-2-carbothioic acid (3-amino-cyclohexyl)-amide
5-(4-Chloro-phenyl)-N-(4,4-difluoro-cyclohexyl)-2-methoxymethyl-6-[4-(3,3,3-trifluoro-propoxy)-phenyl]-thionicotinamide and
5,6-Bis-(4-chloro-phenyl)-3-methoxymethyl-pyridine-2-carbothioic acid (1-hydroxymethyl-3-methyl-butyl) amide;
and pharmaceutically acceptable salts thereof.

10. (canceled)

11. A pharmaceutical formulation comprising a compound of formula I as claimed in claim 1 or claim 9 and a pharmaceutically acceptable adjuvant, diluent or carrier.

12. (canceled)

13. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy and related conditions, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of formula I as claimed in claim 1 to a patient in need thereof.

14. (canceled)

15. A process for the preparation of a compound of formula I according to claim 1 comprising reacting a compound of formula II 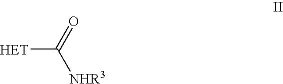
in which Het and R³ are as defined in claim 1 with Lawesson's reagent in the presence of a diluent at a temperature in the range of 50° C. to 250° C.
